(19) United States
(12) Patent Application Publication
(10) Pub. No.: US 2021/0069433 A1
(43) Pub. Date: Mar. 11, 2021

(54) Electronic Inhaling Device

(71) Applicant: Virgilant Technologies Limited, Taipei City (TW)

(72) Inventors: Chung Sheng Wang, Taipei City (TW); Jonathan David Piteo Tarpy, New York City, NY (US)

(21) Appl. No.: 17/024,660

(22) Filed: Sep. 17, 2020

Related U.S. Application Data
(63) Continuation of application No. 15/623,053, filed on Jun. 14, 2017, now abandoned.

(30) Foreign Application Priority Data
Jun. 15, 2016 (TW) 105118757
Jun. 15, 2016 (TW) 105118758

(51) Int. Cl. A61M 15/00

(52) U.S. Cl. CPC A61M 15/009; A61M 15/0073; A61M 15/0066; A61M 15/008; H01H 36/0006; A61M 2205/8206; A61M 2205/3317; A61M 2205/3592; A61M 2205/52; A61M 2205/3561

(57) ABSTRACT

An electronic inhaling device for detachably receiving a spray bottle having a bottle body with a spray mouth, includes: a casing formed with a vertical slide slot, in which the spray bottle is adapted to be disposed slidably; a bottle support seat installed in the casing, having a receiving inlet permitting extension of the spray mouth of the bottle body when the spray bottle is seated on the support seat and a discharging outlet in spatial communication with the receiving inlet in order to discharge a volatile substance sprayed out from the spray mouth of the bottle body via the discharging outlet; and a monitoring module disposed in the casing for monitoring movement of the spray mouth relative to the bottle body, counting a discharged number of the volatile substance sprayed out from the spray mouth of the bottle body via the discharging outlet.

### BACKGROUND OF THE INVENTION

### 1. Field of the Invention

The present invention relates generally to an inhaling device, and more particularly to an electronic inhaling device for an inhaler (such as tracheal dilator), which can let a user to understand the used number or times of the inhaler.

### 2. The Prior Arts

As far as taking medicines is concerned, oral doses are given in addition to hypodermic injection and applying medicine lotion over a patient's skin if the injured part is externally visible. Another taking medicine method is through inhaling, inhaling or spraying in some cases, like tonsillitis or throat pains.

To eliminate or reduce the pains caused by the respiratory system, such as asthma or tonsillitis, inhaling or inhaling method is preferred to hypodermic injection due to the reasons that the in-take medicine or substance can directly react to the respiratory tract rather than any other body part. Therefore, an inhaler or a tracheal dilator is generally carried along with a patient if he is suffering from asthma in addition to the prescribed medicines.

It is noted that a spray bottle containing the liquid medicine (in volatile form) is usually placed within an inhaler or an inhaling device such that upon activation, the liquid medicine is atomized and is sprayed out from the inhaling device in mist formation such that the patient can inhale or swallow the medicine mist via his nose or mouth.

A spray bottle can be used for many times due to its specific amount, but the patient generally cannot remember the number of spraying, and does not know if the liquid medicine still remains in the bottle. If he or she prepares a new one whenever circumstance demands, a waste of medicine may result so does an extra expense.

One serious disadvantage resides in that the spray bottle is already empty, but the patient thinks there is still sufficient amount of content therein. In case he she suffers from asthmatic problem all of a sudden and it requires him to inhale the liquid medicine from the spray bottle immediately to relieve the pain, but there is no medicine available in the spray bottle, then his health is put in a dangerous condition.

### SUMMARY OF THE INVENTION

A primary objective of the present invention is to provide an electronic inhaling device for receiving a spray bottle therein such that the electronic inhaling device includes a monitoring module capable of displaying the discharged or spray-out number of liquid medicine contained in the spray bottle, thereby eliminating the disadvantages encountered during use of conventional spray bottles.

An electronic inhaling device of the present invention for detachably receiving a spray bottle having a bottle body with a supply mouth, includes: a casing formed with a vertical slide slot, in which the spray bottle is adapted to be disposed slidably; a bottle support seat installed in the casing, having a receiving inlet permitting extension of the supply mouth of the bottle body when the spray bottle is seated on the support seat and a discharging outlet in spatial communication with the receiving inlet in order to discharge a volatile substance sprayed out from the spray mouth of the bottle body via the discharging outlet; and a monitoring module disposed in the casing for monitoring movement of the spray mouth relative to the bottle body, counting a discharged number of the substance sprayed out from the spray mouth of the bottle body via the discharging outlet.

In one embodiment of the present invention, the monitoring module in the electronic inhaling device of the present invention further includes a reed switch installed within the casing and a magnetic member disposed movably in the casing along the vertical slide slot in such a manner that movement of the magnetic member toward and away from the reed switch results in activation and de-activation of the reed switch and causing the discharged number of the substance sprayed out from the spray mouth of the bottle body via the discharging outlet.

An important aspect to note in the abovementioned two embodiments of the electronic inhaling device of the present invention is that since the monitoring module can observe the relative movement between the spray mouth and the bottle body, hence the discharged number of the content sprayed out from the spray mouth of the bottle body is precisely counted. Thus, the user can definitely know the remaining portion of liquid medicine within the bottle body and the remaining number of dosages. In other words, he can adjustably prepare a new one for replacement without causing medicine waste and extra expense. The problem of running out of medicine without noticing can be avoided.

### BRIEF DESCRIPTION OF THE DRAWINGS

The present invention will be apparent to those skilled in the art by reading the following detailed description of a preferred embodiment thereof, with reference to the attached drawings, in which:

FIG. 1A is a perspective view of an electronic inhaling device according to a first embodiment of the present invention;

FIG. 1B is a perspective view of the electronic inhaling device according to the first embodiment of the present invention shown in FIG. 1A from another angle;

FIG. 2 is a fragmentary sectional view of the electronic inhaling device according to the first embodiment of the present invention shown in FIG. 1A;

FIG. 3A is a partially sectional view illustrating the electronic inhaling device of the present invention shown in FIG. 1A;

FIG. 3B is a front and partially sectional view illustrating the electronic inhaling device of the present invention shown in FIG. 1A;

FIGS. 4A, 5A, 6A, 7A, 8A and 9A are partially sectional views of FIG. 3A respectively illustrating activation of a spray bottle within a casing in the electronic inhaling device of the present invention;

FIGS. 4B, 5B, 6B, 7B, 8B and 9B are front and partially sectional views of FIG. 3B respectively illustrating activation of a spray bottle within a casing in the electronic inhaling device of the present invention;

FIG. 10A is a perspective view of an electronic inhaling device according to a second embodiment of the present invention;

FIG. 10B is a perspective view of the electronic inhaling device according to the second embodiment of the present invention from another angle;

FIG. 11 is a perspective and exploded view of the electronic inhaling device of the present invention shown in FIG. 10A;

FIG. 12 is a partially sectional view of the electronic inhaling device of the present invention shown in FIG. 10A;

FIGS. 13A, 14A, 15A and 16A are partially sectional views of FIG. 12 respectively illustrating activation of a spray bottle within a casing in the electronic inhaling device of the present invention; and

FIGS. 13B, 14B, 15B and 16B are fragmentary views of FIG. 12 respectively illustrating a driving roller meshed with a plurality of stationary teeth of a bottle support seat in the electronic inhaling device of the present invention.

### DETAILED DESCRIPTION OF THE PREFERRED EMBODIMENTS

The accompanying drawings are included to provide a further understanding of the invention, and are incorporated in and constitute a part of this specification. The drawings illustrate embodiments of the invention and, together with the description, serve to explain the principles of the invention.

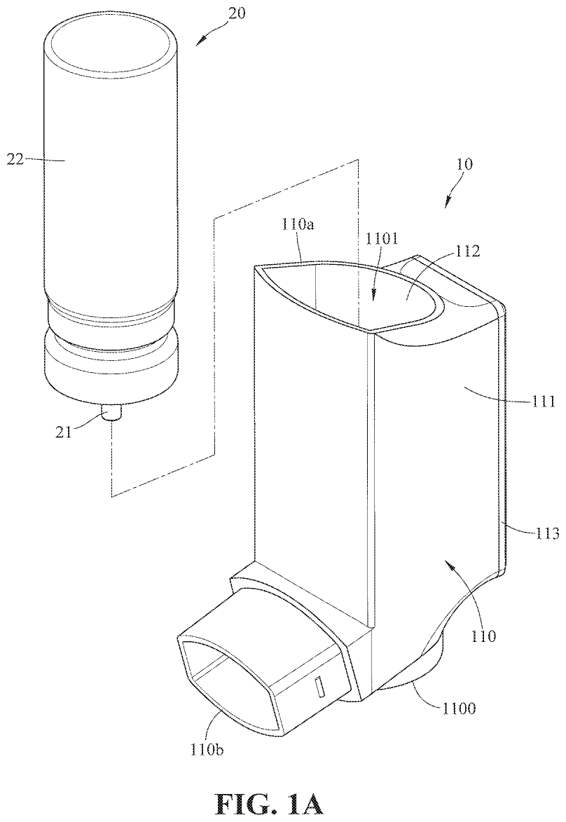

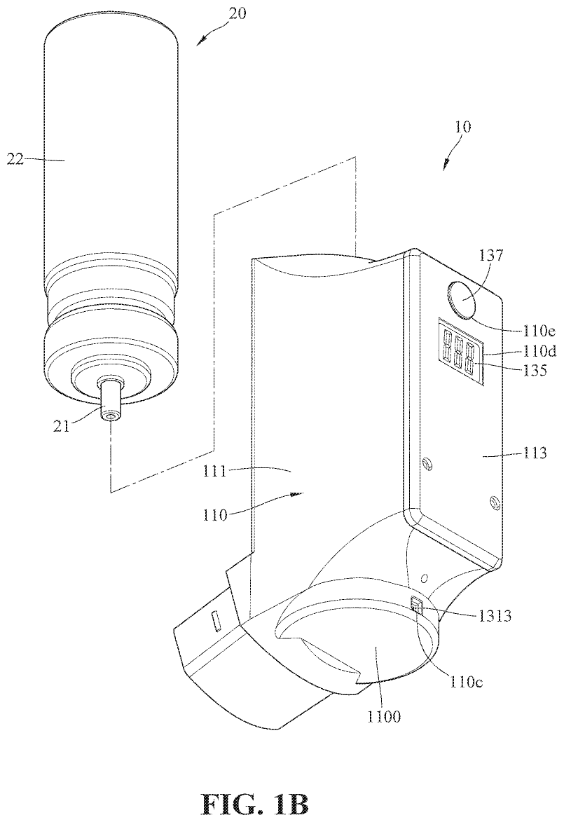

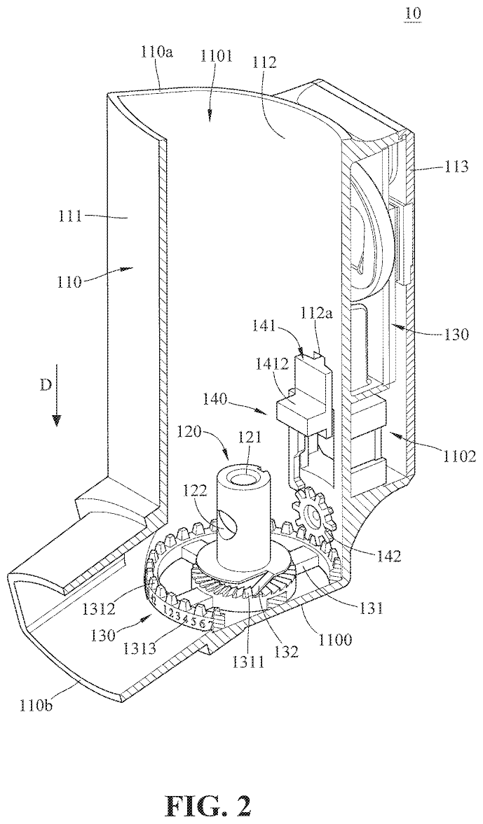

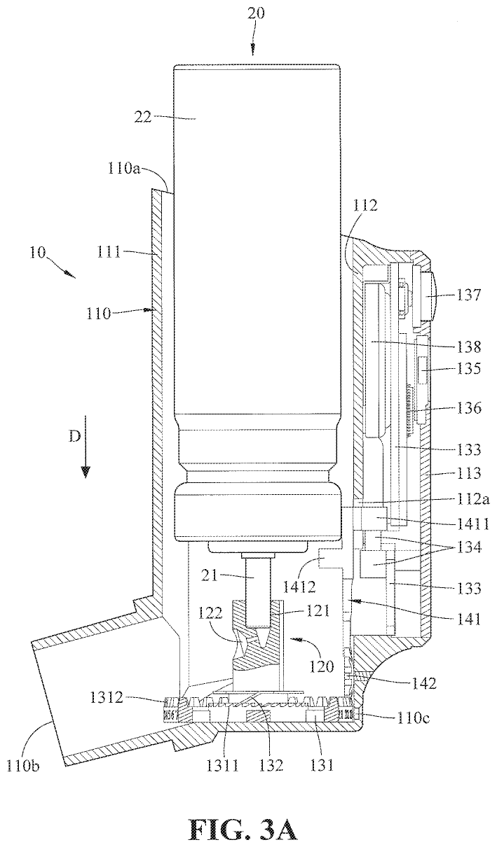

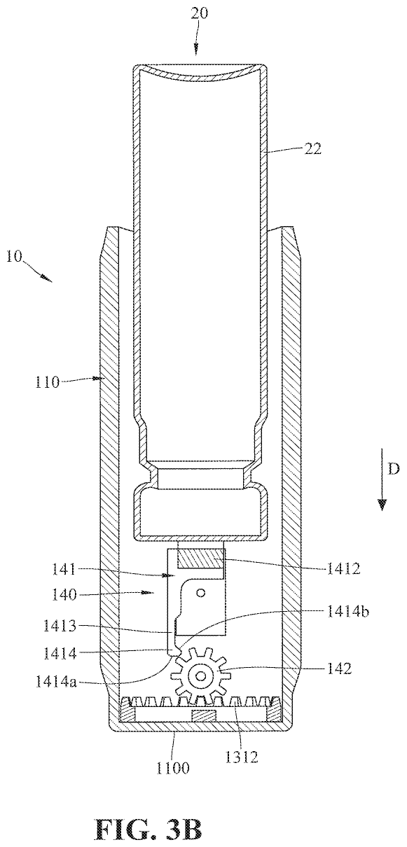

Referring to FIGS. 1-3, wherein FIG. 1A is a perspective view of an electronic inhaling device according to a first embodiment of the present invention; FIG. 1B is a perspective view of the electronic inhaling device according to the first embodiment of the present invention shown in FIG. 1A from another angle; FIG. 2 is a fragmentary sectional view of the electronic inhaling device according to the first embodiment of the present invention shown in FIG. 1A; FIG. 3A is a partially sectional view illustrating the electronic inhaling device of the present invention shown in FIG. 1A; and FIG. 3B is a front and partially sectional view illustrating the electronic inhaling device of the present invention shown in FIG. 1A.

As best shown in FIGS. 1A and 1B, an electronic inhaling device 10 of the present invention is adapted for detachably receiving a spray bottle 20 having a bottle body 22 with a spray mouth 21, wherein the spray mouth 21 is movable relative to the bottle body 22 between a retracted position, in which the spray mouth 21 is retracted inwardly relative to the bottle body 22, where the volatile substance (or liquid medicine) within the bottle body 22 is compressed such that the substance is discharged from the spray mouth 21 and a normal position, in which the spray mouth 21 extends outwardly and axially from the bottle body 22.

As shown in FIG. 2, the electronic inhaling device 10 of the present invention includes a casing 110, a bottle support seat 120, a monitoring module 130, and a linkage module 140.

Referring to FIGS. 2, 3A and 3B, the casing 110 includes a main casing 111 and a partition wall 112 disposed in the main casing 111 to define a first chamber 1101 and a second chamber 1102, wherein the partition wall 112 is formed with a vertical slide slot 112a extending along a moving direction D and spatially communicating the first and second chambers 1101, 1102. Preferably, the main casing 111 defines a bottom surface 1100, an insertion opening 110a via which the spray bottle 20 can be inserted into the first chamber 1101, a discharging opening 110b and a viewing window 110c formed adjacent to the bottom surface 1100, the purpose of which will be given later. The second chamber 1102 has an external wall 113 formed with a press button 137 and a display opening 110d below the press button 137.

Referring to FIGS. 2, 3A and 3B, the bottle support seat 120 is installed integrally or detachably on the bottom surface 1100 of the first chamber 1101 of the casing 110, has a receiving inlet 121 permitting hermetically extension of the spray mouth 21 of the bottle body 22 when the spray bottle 20 is seated thereon and a discharging outlet 122 in spatial communication with the receiving inlet 12 in order to discharge a volatile substance sprayed out from the spray mouth 21 of the bottle body 22 via the discharging outlet 122. Note the receiving inlet 121 is oriented toward the insertion opening 110a of the first chamber 1101 while the discharging outlet 122 is oriented toward the discharging opening 110b of the main body 111, but should not be limited only to the disclosed ones. Any other means so long as the functions can be accomplished should be encompassed.

Referring to FIG. 3A, the monitoring module 130 includes a marked wheel 131, a position limiting element 132, a printed circuit board 133, a spring-biased switch 134, a display panel 135, a communication unit 136, a press button 137 and a battery unit 138.

The marked wheel 131 is disposed rotatably and co-axially within the casing 110 around the bottle support seat 120, and has a plurality of ratchet teeth 1311 circumferentially formed on an inner side, a plurality of rotation teeth 1312 circumferentially formed around the ratchet teeth 1311 and a plurality of marked patterns 1313 formed on an outer peripheral surface thereof. The position limiting element 132 is fixed to the bottle support seat 120 so as to be meshed one of the plurality of ratchet teeth 1311.

Because each ratchet teeth 1311 has two different inclined sides such that the marked wheel 131 is rotatable about its axis only in a single direction. In other words, the marked wheel 131 is not rotatable about its axis opposite to the single direction. The marked patterns 1313 may be digit numbers such that rotation the marked wheel 131 about its axis in the single direction displays a specific one of the marked patterns via a viewing window 110c formed through the casing 110. The specific one of the marked patterns 1313 defines the discharged number of the substance sprayed out from the spray mouth 21 of the bottle body 22 via the discharging outlet 122. Preferably, a unidirectional bearing unit may serve the purpose of the position limiting element 132.

Referring again to FIG. 3A, in this embodiment, the printed circuit board 133, the spring-biased switch 134, the display panel 135, the communication unit 136, the press button 137 and the battery unit 138 are mounted in the second chamber 1102 of the casing 110 in such a manner that all these elements are connected electrically relative to one another via the printed circuit board 133 or connection wires (not visible) so as to permit movement of the spray bottle 20 along the moving direction D or relative movement between the spray mouth 21 and the bottle body 22 such that the spring-biased switch 134 is installed within the second chamber 1102 of the casing 110 so as to be activated upon contacting with the bottle body 22 when the latter moves along the moving direction D for counting the discharged number of the substance from the bottle body 22 via the discharging outlet 122.

Referring to FIGS. 1B and 3A, the display panel 135 is oriented toward the display opening 110d for displaying the discharged number of the substance from the bottle body 22. The communication unit 136 is used for transmission of the information to an electronic device (not visible) located exterior of the casing 110. The information may include serial number of the spray bottle 20 and the discharged number of the substance sprayed out from the spray mouth 21 of the bottle body 22. The press button 137 is exposed from a press opening 110e formed through the external wall 113 of the second chamber 1102, pressing of which activates the communication unit 136. The battery unit 138 supplies electrical power the monitoring module 130. The electronic device (not visible) is installed with a monitoring program utilized by the communication unit 136 for transmitting the information including the discharged number of the substance sprayed out from the spray mouth 21 of the bottle body 22 and a schedule time for replacing the spray bottle with a new ones.

Referring to FIG. 3B, the linkage module 140 includes a pushing element 141 and a driving gear 142 that is disposed rotatably in the casing 110 and that meshes with the plurality of rotation teeth 1312 of the marked wheel 131. Preferably, the driving gear 142 defines a rotating axis transverse to the co-axis of the mark wheel 131 and the bottle support seat 120.

The pushing element 141 has a pressing part 1411 extending into the second chamber 1102 via the vertical slide slot 112a and a compressed part 1412 that is connected integrally with the pressing part 1411, that supports the bottle body 22 thereon and that is disposed within the first chamber 1101 such that movement of the linkage module 140 together with the bottle body 22 along the vertical slide slot 112a in the moving direction D results in collision of the pressing part 1411 of the linkage module 140 against the spring-biased switch 134 and simultaneous movement of the driving part 1414 and driving the gear 142, thereby rotating the marked wheel 131 about its axis in the single direction and displaying a specific one of the marked patterns 1313 via a viewing window formed through the casing 110

In this embodiment, the pushing element 141 of the linkage module 140 further includes a neck part 1413 interconnecting the compressed part 1412 and the driving part 1414 having a cross section smaller than the compressed part 1412 and the driving part 1414 so as to provide resiliency.

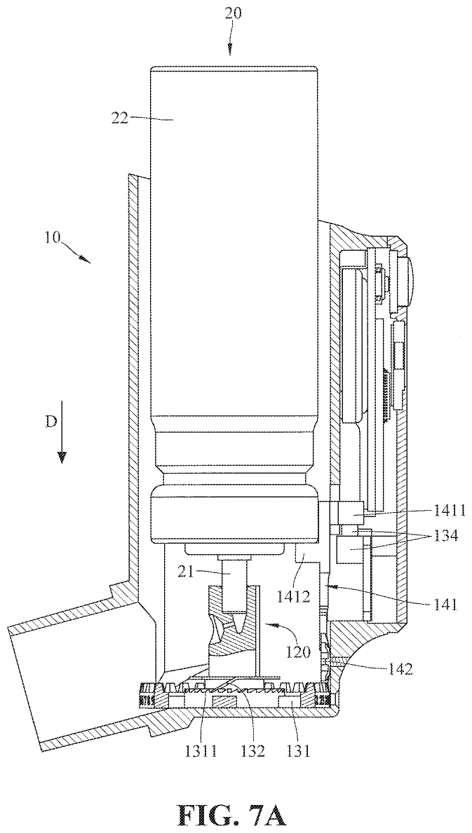

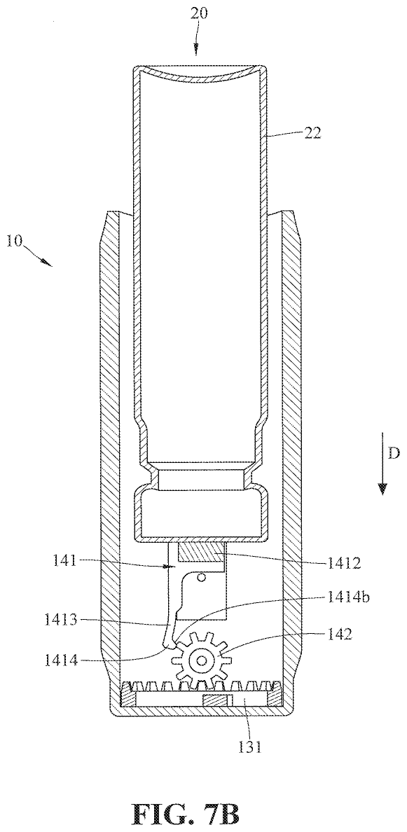

The driving part 1414 has a flat abutment face 1414a that abuts against the driving gear 142 when the driving part 1414 is moved along the moving direction D of the bottle body 22, where the driving part 1414 and the neck part 1413 are proximate to an axis of the gear 142 (see FIG. 3B) and an inclined abutment face 1414b that abuts against the driving gear 143 when the driving part 1414 is moved opposite to the moving direction D, where the assembly of the neck part 1413 and the driving part 1414 is bent relative to the compressed part 1412 and are disposed distal to an axis of the gear 142 due to the single direction rotation of the marked wheel 131 being meshed with the driving gear 142 (see FIG. 7B). At this time, the driving part 1414 of the pushing element 141 can disengage from the driving gear 142 if requires, like removing the spray bottle 20 from the casing 110 for replacing with a new one.

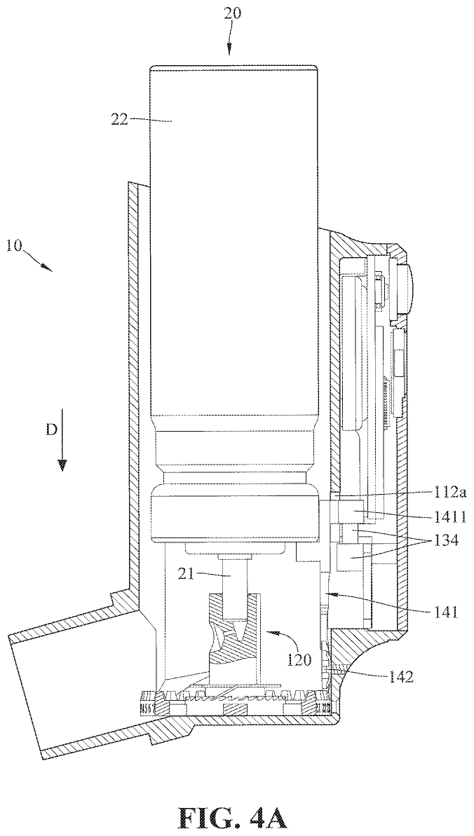

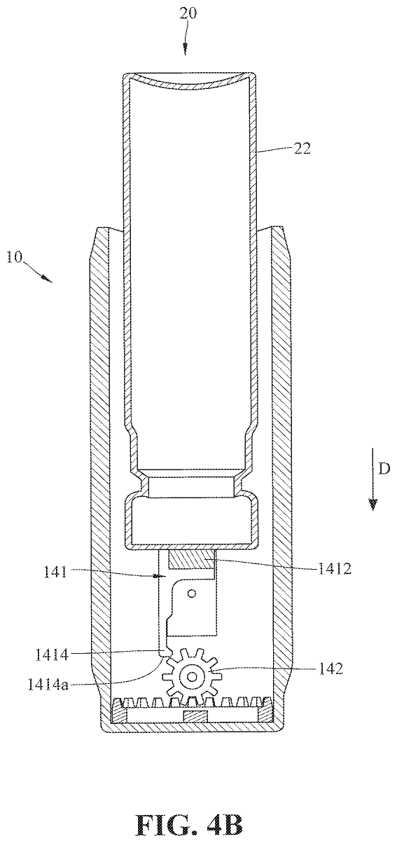

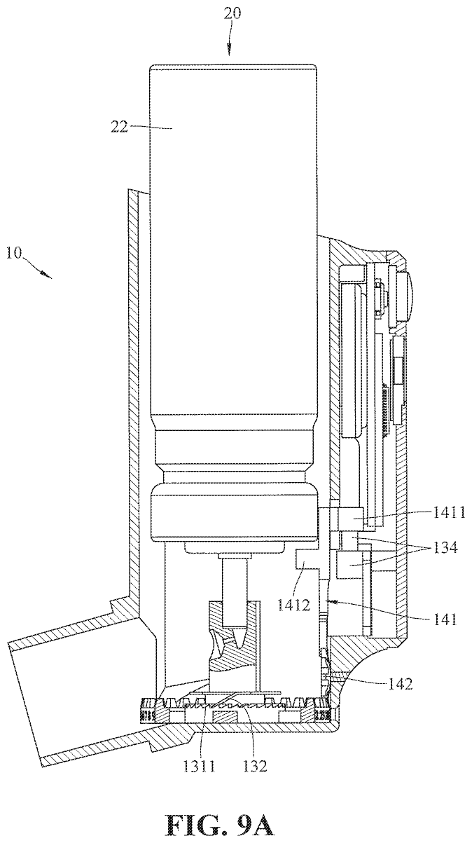

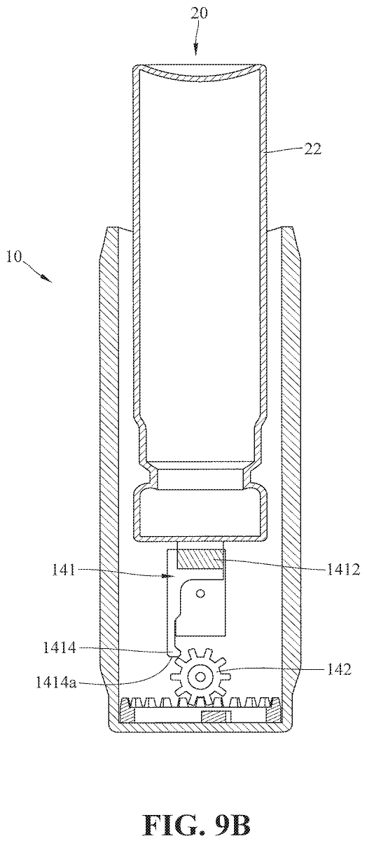

FIGS. 4A, 5A, 6A, 7A, 8A and 9A are partially sectional views of FIG. 3A respectively illustrating activation of a spray bottle 20 within a casing 110 in the electronic inhaling device 10 of the present invention while FIGS. 4B, 5B, 6B, 7B, 8B and 9B are front and partially sectional views of FIG. 3B respectively illustrating activation of a spray bottle within a casing in the electronic inhaling device of the present invention.

Referring again to FIGS. 3A and 3B, wherein the spray mouth 21 is movable relative to the bottle body 22 between a normal position, in which the spray mouth 21 extends outwardly and axially from the bottle body 22 and in which the bottle body 22 does not contact with the compressed part 1412 of the pushing element 141 such that the pressing part 1411 slightly contacts the spring-biased switch 134 without activating the spring-biased switch 134. At this time, the flat abutment face 1414a of the driving part 1414 neither contact nor drive the driving gear 142 while the position limiting element 132 engages one of the plurality of ratchet teeth 1311.

Referring to FIGS. 4A and 4B, when the user needs the spray bottle 20 to spray out the substance from the spray mouth 21, movement of the bottle body 22 along the moving direction D against the bottle support seat 120 in a first distance results in contact of the bottle body 22 with the compressed part 1412 so that the spray mouth 21 retracted into the bottle body 22 and compressing the substance, where the pressing part 1411 contacts but does not activate the spring-biased switch 134 while the flat abutment face 1414a of the driving part 1414 does not contact the driving gear 142.

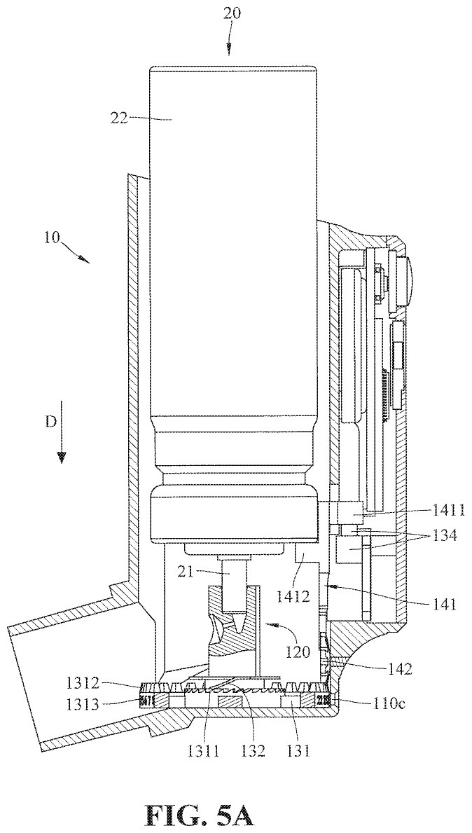

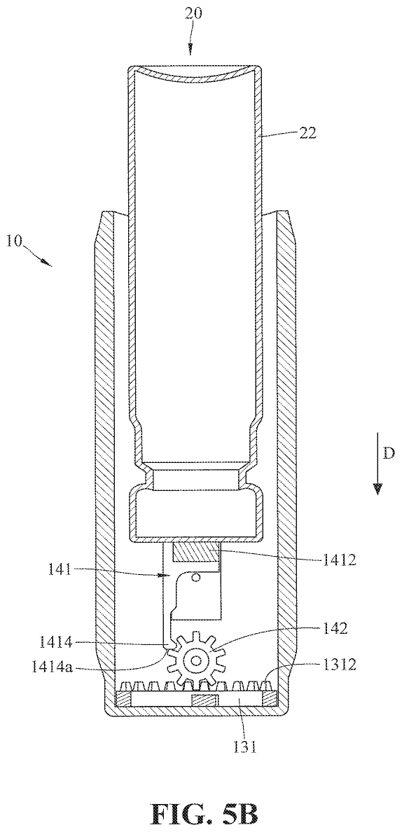

Referring to FIGS. 5A and 5B, movement of the bottle body 22 along the moving direction D against the bottle support seat 120 from the first distance toward a second distance results in the contact of the bottle body 22 with the compressed part 1412 so that the spray mouth 21 retracted into the bottle body 22 and compressing the substance to spray out the substance from the spray mouth 21, where the pressing part 1411 compresses but does not activate the spring-biased switch 134 while the flat abutment face 1414a of the driving part 1414 drives the driving gear 142 to rotate about its axis in a half angle of the gear teeth, where the marked wheel 131 is rotated about its axis in a half angle of one rotation teeth 1312, thereby displaying a specific one of the marked patterns 1313 via the viewing window 110c of the casing 110.

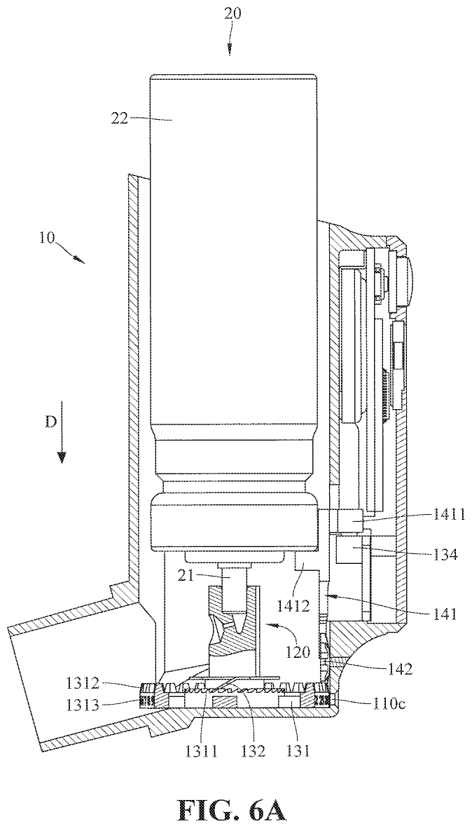

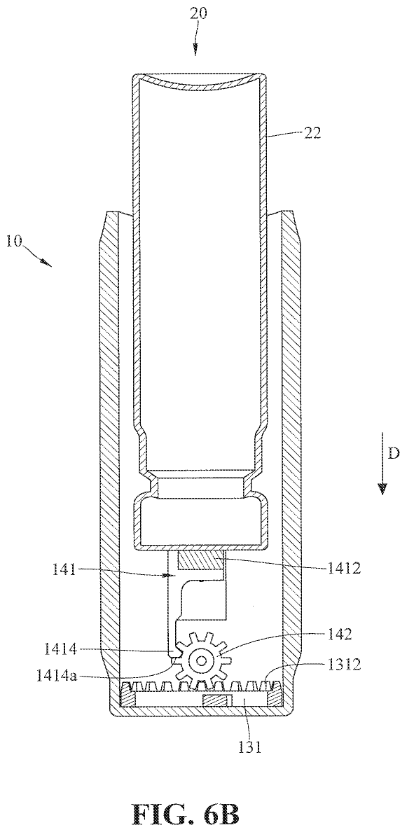

Referring to FIGS. 6A and 6B, movement of the bottle body 22 along the moving direction D against the bottle support seat 120 results in the contact of the bottle body 22 with the compressed part 1412 so that the spray mouth 21 retracted into the bottle body 22 and compressing the substance and spraying out the substance via the spray mouth 21, where the pressing part 1411 compresses and activates the spring-biased switch 134 while the flat abutment face 1414a of the driving part 1414 drives the driving gear 142 to rotate about its axis in another half angle of the gear teeth compared with FIGS. 3A and 3B, where the marked wheel 131 is rotated about its axis in another half angle of one rotation teeth 1312, thereby displaying another specific one of the marked patterns 1313 via the viewing window 110c of the casing 110. Note that the another specific marked pattern 1313 includes one more spraying action of the substance from the spray mouth 21.

Referring to FIGS. 7A and 7B, the spray mouth 21 is movable relative to the bottle body 22 from the retracted position to a normal position, in which the spray mouth 21 extends outwardly and axially from the bottle body 22. Because, the spray mount 21 is seated on the bottle support seat 120, the bottle body 22 and the pressing part 1411 move in the direction opposite of the moving direction D due to restoration force of freeing the spring-biased switch 134 from being compressed such that the compressed part 1412 lifts the bottle body 22 upward in a first space interval. However, the upward movement of the bottle body 22 is limited due to meshing of the ratchet teeth 1311 of the marked wheel 131 with the position limiting element 132, thereby preventing rotation of the marked wheel 131 in a direction opposite to the single direction. So does the driving gear 142 such that an assembly of the driving part 1414 and the neck part 1413 is bent relative to the compressed part 1412 and are disposed distal to an axis of the gear 142. At this time, the driving part 1414 can disengage from the driving gear 142 if requires.

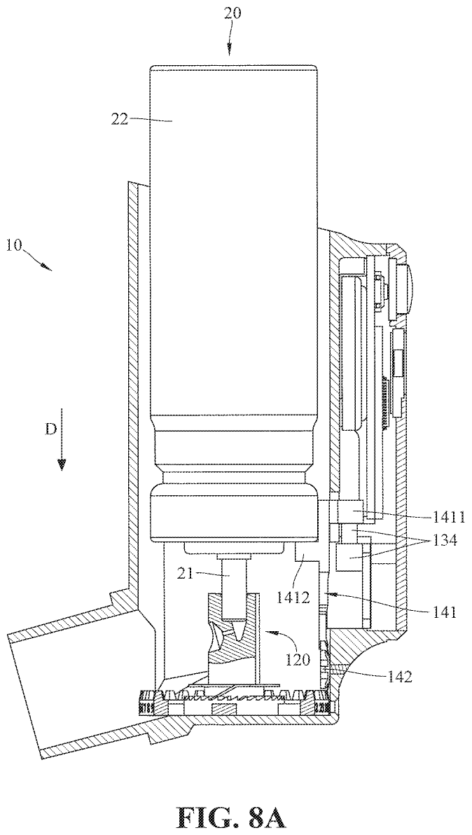

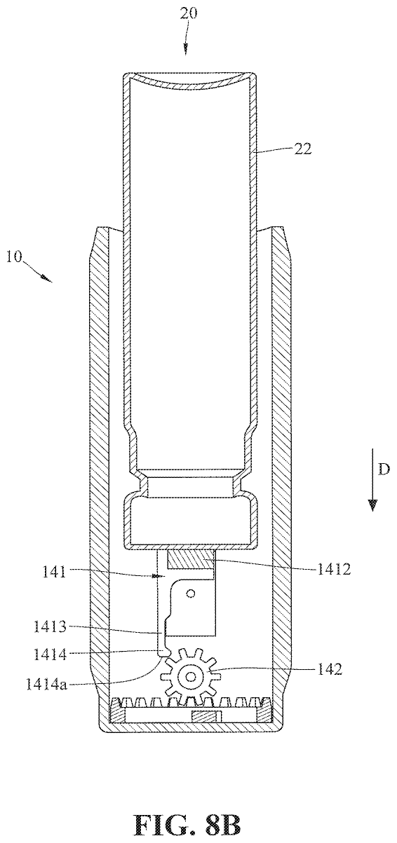

Referring to FIGS. 8A and 8B, movement of the bottle body 22 in the opposite direction from the first space interval toward a second space interval results in totally freeing the spring-biased switch 134 from being compressed such that the compressed part 1412 lifts the bottle body 22 upward to the second space interval, where the spring-biased switch 134 contacts the pressing part 1411 such that the driving part 1414 and the neck part 1413 are proximate to an axis of the gear 142 so that the flat abutment face of 1414a of the driving part 1414 does contact with the driving gear 142.

Note in one embodiment of the present invention, since the electronic or mechanical elements are employed to count the discharged number of the substance from the spray bottle 20, the marked patterns 1313 of the spray bottle 20 can be visible via the viewing window 110c in the casing 110, thereby understanding the discharged number of the spray bottle 20. In the event that the information of the electronic inhaling device of the present invention is required to be transmitted to an electronic device, like a smart phone of a user, the user can press the button 137 to activate the communication device 136, which transmits the information of the spray bottle 20 to the smart phone. Preferably, the smart phone has a monitoring program utilized by the communication unit 136 for transmitting the discharged number of the substance sprayed out from the spray mouth 21 of the bottle body 22 via the discharging outlet 122.

Of course in the abovementioned embodiment, since the user can know the discharged number of the spray bottle 20, he or she can predict the remaining amount of the substance in the spray bottle 20, and hence a schedule time for replacing the spray bottle with a new one, thereby saving and economizing the medicine waste.

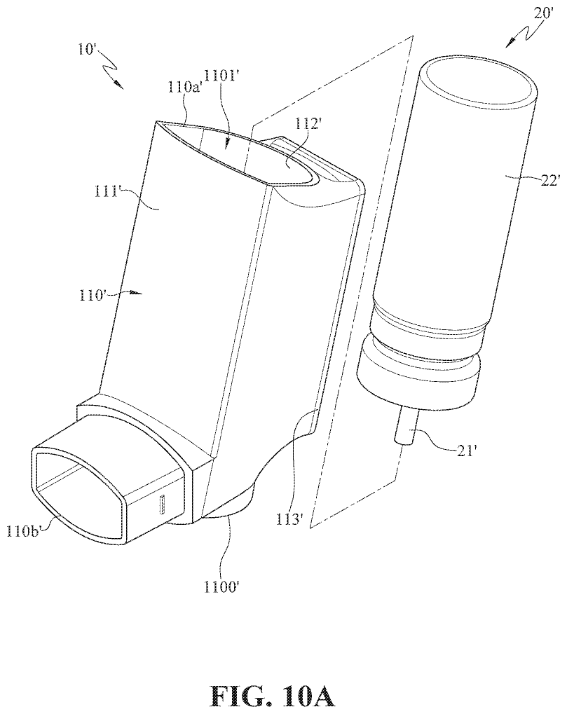

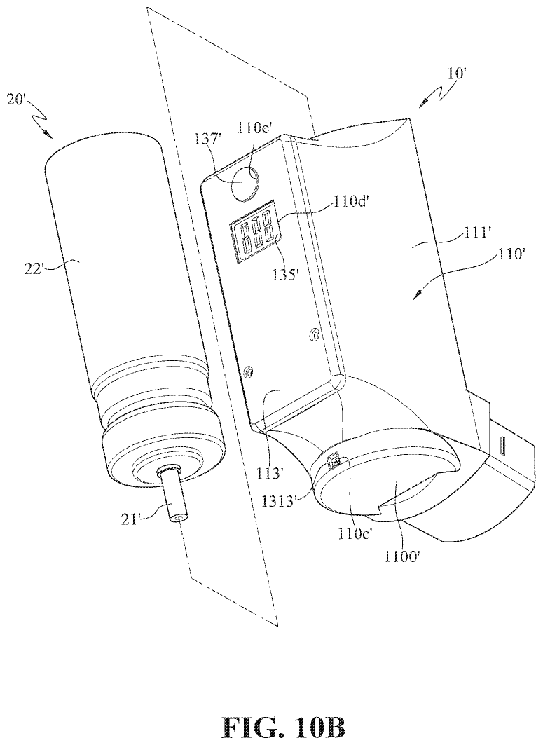

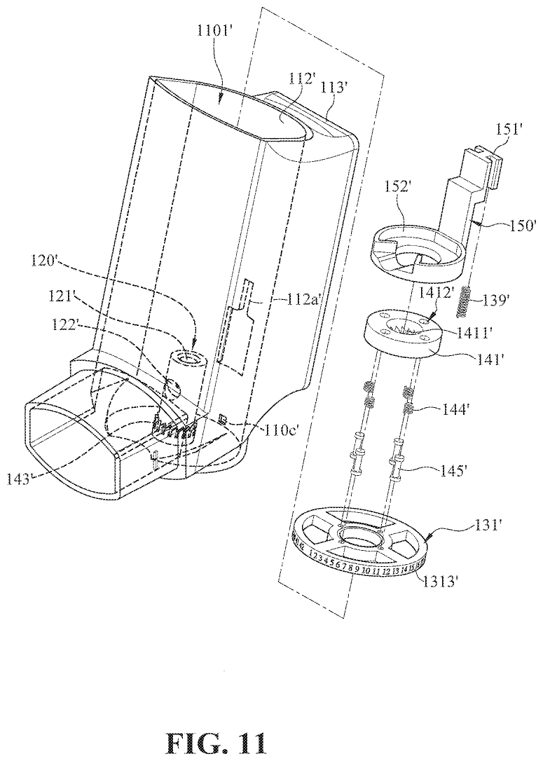

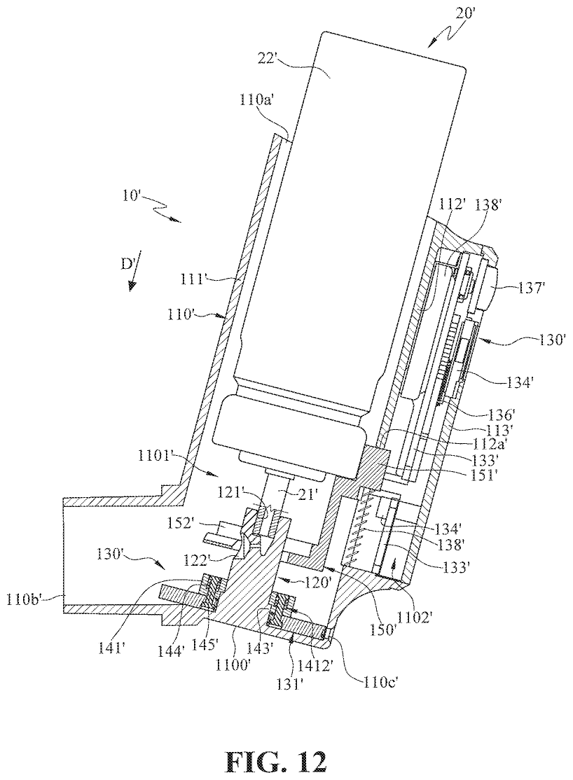

Referring to FIGS. 10A-12, wherein FIG. 10A is a perspective view of an electronic inhaling device 10′ according to a second embodiment of the present invention; FIG. 10B is a perspective view of the electronic inhaling device 10′ according to the second embodiment of the present invention from another angle; FIG. 11 is a perspective and exploded view of the electronic inhaling device 10′ of the present invention shown in FIG. 10A; and FIG. 12 is a partially sectional view of the electronic inhaling device 10′ of the present invention shown in FIG. 10A;

As best shown in FIGS. 10A and IOB, similar to the previous embodiment, in the second embodiment of the present invention, the spray mouth 21′ is movable relative to the bottle body 22′ between a retracted position, in which the spray mouth 21′ retracted inwardly relative to the bottle body 22′, where the substance within the bottle body 22′ is compressed and is discharged from the spray mouth 21′, and a normal position, in which the spray mouth 21′ extends outwardly and axially from the bottle body 22′.

Referring to FIG. 11, the electronic inhaling device 10′ according to the second embodiment of the present invention includes a casing 110′, a bottle support seat 120′ and a monitoring module 130′.

The casing 110′ includes a main casing 111′ and a partition wall 112′ disposed in the main casing 111′ to define a first chamber 1101′ and a second chamber 1102′, wherein the partition wall 112′ is formed with a vertical slide slot 112a′ extending along a moving direction D′ and spatially communicating the first and second chambers 1101′, 1102′. Preferably, the main casing 111′ defines a bottom surface 1100′, an insertion opening 110a′ via which the spray bottle 20′ can be inserted into the first chamber 1101′, a discharging opening 110b′ and a viewing window 110c′ formed adjacent to the bottom surface 1100′.

The bottle support seat 120′ is installed integrally or detachably on the bottom surface 1100′ of the first chamber 1101′ of the casing 110, has a receiving inlet 121′ permitting hermetically extension of the spray mouth 21′ of the bottle body 22′ when the spray bottle 20′ is seated on the support seat 120′ and a discharging outlet 122′ in spatial communication with the receiving inlet 12′ in order to discharge a volatile substance sprayed out from the spray mouth 21′ of the bottle body 22′ via the discharging outlet 122′. Note the receiving inlet 121′ is oriented toward the insertion opening 110a′ of the first chamber 1101′ while the discharging outlet 122′ is oriented toward the discharging opening 110b′ of the main body 111′.

Referring to FIG. 11, the monitoring module 130′ includes a magnetic member 150′, a printed circuit board 133′, a reed switch 134′, a display panel 135′, a communication unit 136′, a press button 137′, a battery unit 138′, a marked wheel 131′, a driving roller 141′ formed with a plurality of ratchet teeth 1411′, and a plurality of rotation teeth 143′.

The reed switch 134′ is installed within the second chamber 1102′ of the main casing 111′. The magnetic member 150′ is fabricated from magnetic materials, has a sensing section 151′ extending into the second chamber 1102′ through the vertical slide slot 112a′ and an attraction section 152′ disposed in the first chamber 1101′ so as to support the bottle body 22′ thereon. A biasing unit 139′ is disposed between the magnetic member 150′ and the casing 110′.

In this embodiment, the printed circuit board 133′, the reed switch 134′, the display panel 135′, the communication unit 136′, the press button 137′ and the battery unit 138′ are installed in the second chamber 1102′ of the casing 110′. The reed switch 134′, the display panel 135′, the communication unit 136′, the press button 137′ and the battery unit 138′ are electrically connected relative to one another via multiple printed circuit boards or wire connection. The magnetic member 150′ is movable along the vertical slide slot 112a′ in such a manner that movement of the sensing section 151′ of the magnetic member 150′ toward and away from the reed switch 134 (or relative movement between the spray mouth 21′ and the bottle body 22′) results in activation and de-activation of the reed switch 134′ and causing the discharged number of the substance sprayed out from the spray mouth 21′ of the bottle body 22′ via the discharging outlet 122′.

Preferably, the display panel 135′ is oriented toward the display opening 110d′ for displaying the discharged number of the spray bottle 20. The communication unit 136′ is used for transmission of the information to an electronic device (not visible) located exterior of the casing 110′. The information may include serial number of the spray bottle 20′ and the discharged number of the substance sprayed out from the spray mouth 21′ of the bottle body 22′. The press button 137′ is exposed from a press opening 110e′ formed through the external wall 113′ of the second chamber 1102′, pressing of which activates the communication unit 136′. The battery unit 138′ supplies electrical power the monitoring module 130′. The electronic device (not visible) is installed with a monitoring program utilized by the communication unit 136′ for transmitting the information including the discharged number of the substance sprayed out from the spray mouth 21′ of the bottle body 22′ and a schedule time for replacing the spray bottle with a new ones.

Referring to FIGS. 11 and 12, the driving roller 141′ formed with a plurality of inner ratchet teeth 1411′ and the marked wheel 131′ having a plurality of marked patterns 1313′ formed on an outer peripheral surface are disposed co-axially in the first chamber 1101′ around the bottle support seat 120′ in such manner that the inner ratchet teeth 1411′ of the driving roller 141′ meshed with the stationary teeth 143′ of the bottle support seat 120′, wherein the driving roller 141′ is sandwiched between the marked wheel 131′ and the magnetic member 150′.

Preferably, the driving roller 141′ is fabricated from magnetic materials, like Iron, cobalt, nickel, defines a plurality of through holes 1412′ with different diameters hole sections, in which a plurality of springs 144′ are disposed and a plurality of pins 145′ extend through the springs 144′ in such a manner to resiliently connect the driving roller 141′ and the marked wheel 131′ to permit co-rotation of the driving roller 141′ together with the marked wheel 131′ in the single direction.

Referring to FIGS. 11 and 12, movement of the magnetic member 150′ along the moving direction D′ results in attracting the driving roller 141′ by the attraction section 152′ and movement of the magnetic member 150 in a direction opposite to the moving direction D′ results in limited disengagement between the inner ratchet teeth 141′ of the driving roller 141′ and the stationary teeth 143′ of the support seat 12 by virtue of the pins 145′ and the springs 144′ and permitting limited rotation of the marked wheel 131′ relative to the driving roller 141′, thereby displaying a specific one of the marked patterns 1313′ via the viewing window 110c′.

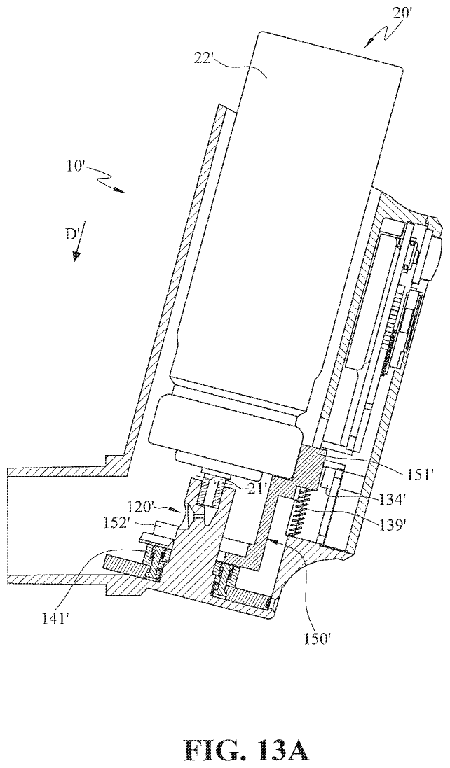

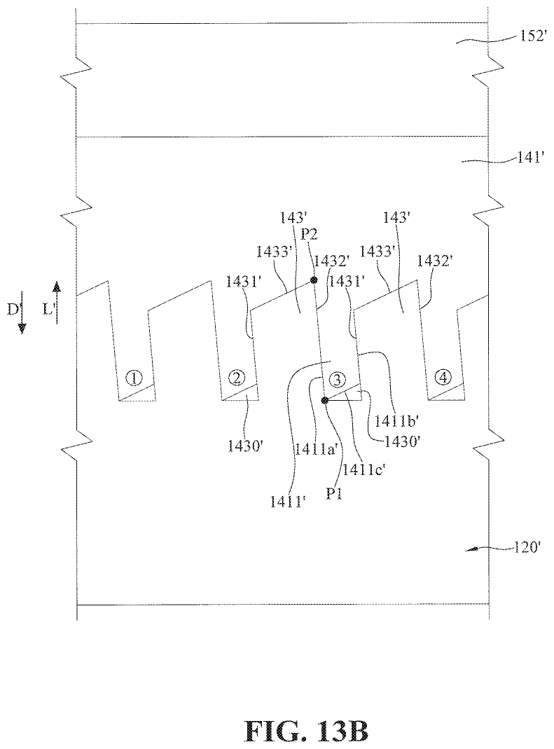

FIGS. 13A, 14A, 15A and 16A are partially sectional views of FIG. 12 respectively illustrating activation of a spray bottle 20′ within a casing 110′ in the electronic inhaling device of the present invention; and FIGS. 13B, 14B, 15B and 16B are fragmentary views of FIG. 12 respectively illustrating a driving roller 141′ meshed with a plurality of stationary teeth 143 of a bottle support seat 120 in the electronic inhaling device of the present invention, wherein the encircled numerals respectively represent the ratchet teeth 1411′ of the driving roller 141′ and the stationary teeth 143′ of the bottle support seat 120′.

As best shown in FIG. 12, at the normal position, the spray mouth 21′ extends outwardly and axially from the bottle body 22′, where the bottle body 22′ abuts against the magnetic member 150′ due to biasing action of the spring 139′ such that the sensing section 151′ of the magnetic member 150′ is located at a first sensing position of the reed switch 134′, where the attracting section 152′ is slightly spaced apart from the driving roller 141′. However, the ratchet teeth 1411′ of the driving roller 141′ are meshed with the stationary teeth 143′ of the support seat 120 by virtue of the biasing spring units 144′, thereby disposing the driving roller 141′ adjacent to but not driving the marked wheel 131′ such that a specific one of the marked patterns 1313′ is visible from the viewing window 110c′ in the casing 110′.

Referring to FIGS. 13A and 13B, when it is desired to spray out the substance from the spray bottle 20′, the spray mouth 21 is pressed downward and is shifted from the normal position to the retracted position, where the substance within the bottle body 22′ is compressed due to movement of the bottle body 22 against the support seat 120 along the moving direction D′ such that the substance is sprayed out via the spray mouth 21′. Since the magnetic member 150′ moves simultaneously with the bottle body 22′ against biasing action of the biasing unit 139′, the sensing section 151′ is shifted to a second sensing position of the reed switch 134′, where the attraction section 152′ attracts the driving roller 141′ without interruption meshing the ratchet teeth 1411′ of the driving roller 141′ with the stationary teeth 143′ of the support seat 120′.

FIG. 13B illustrates a meshing condition between the ratchet teeth 1411′ of the driving roller 141′ and the stationary teeth 143′ of the support seat 120′. Two adjacent stationary teeth 143′ define a gap 1430′ therebetween. Each of the stationary teeth 143′ has two lateral sides 1431′, 1432′ and a top side 1433′ interconnecting the lateral sides 1431, 1432′ and inclined with respect to the moving direction D′. Note that the lateral sides 1431′, 1432′ are nearly parallel with the moving direction D′, however the inclination shown presently are given for illustration purpose only and the lengths thereof are different from each other. One of the lateral sides 1431′, 1432′ and the top side 1433′ cooperatively define an apex P2 for every single one of the stationary teeth 143′.

In the similar manner, each of the ratchet teeth 1411′ has two lateral sides 1411a′, 1411b′ and a bottom side 1411c′ interconnecting the lateral sides 1411a′, 1411b′ and inclined with respect to the moving direction D′. Note that the lateral sides 1411a′, 1411b′ have different lengths. One of the lateral sides 1411a′, 1411b′ and the bottom side 1411c′ cooperatively define an apex P1 of every single one of the ratchet teeth 1411′. When the ratchet teeth 1411′ of the driving roller 141′ are meshed with the stationary teeth 143′ of the support seat 120′, the apex P1 of each of the ratchet teeth 1411′ extends into the gap 1430′ defined between adjacent stationary teeth 143′, where one lateral side 1411b′ of the ratchet teeth 1411′ abuts against one lateral side 1431′ of the stationary teeth 143′ while the other lateral side 1411a′ of the ratchet teeth 1411′ abuts against the other lateral side 1432′ of the stationary teeth 143′ such that the lateral side 1411b′ of the ratchet teeth 1411′ is slightly interfered with the lateral side 1431′ of the stationary teeth 143′ along the moving direction D′.

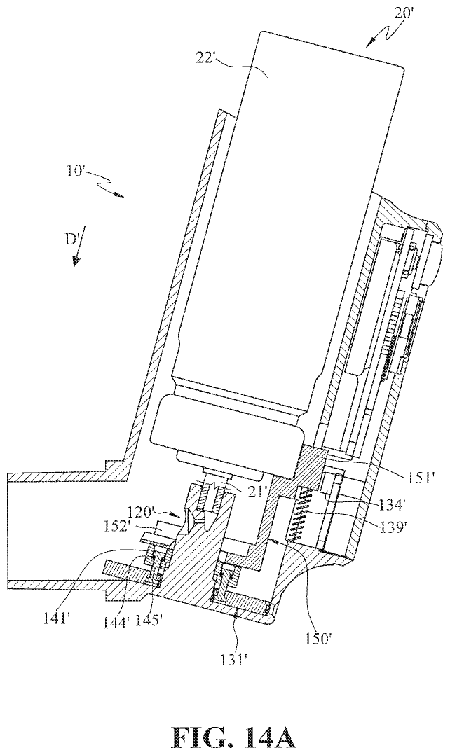

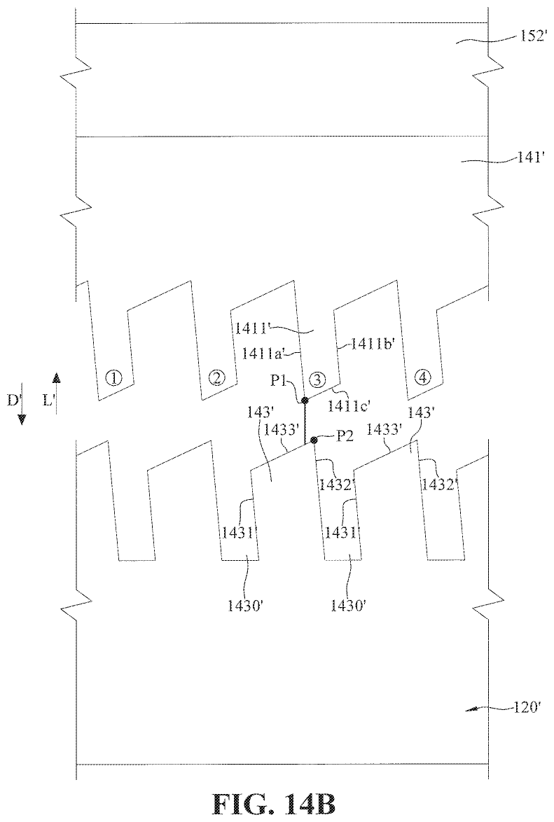

Referring to FIGS. 14A and 14B, during moving the spray mouth 21′ relative to the bottle body 22′ from the retracted position to the normal position, where the spray mouth 21′ extends outwardly and axially from the bottle body 22′ and simultaneously causing the bottle body 22′ moves opposite to the moving direction D′ away from the bottle support seat 120′, which performance is resulted due to restoration force of the biasing unit 139′ such that the sensing section 151′ retrieves its first sensing position of the reed switch 134′ and the driving roller 141′ disengages the marked wheel 13P due to moving away of the attracting section 152′.

As described above, because the lateral side 1411b′ of the ratchet teeth 1411′ is slightly interfered with the lateral side 1431′ of the stationary teeth 143′ along the moving direction D′, movement of the lateral side 1411b′ of the ratchet teeth 1411′ relative to the lateral side 1431′ of the stationary teeth 143′ in the opposite direction L of the moving direction D′ causes disengagement between the ratchet teeth 1411′ and the stationary teeth 143′ and simultaneously results in an inertia for driving of the driving roller 141′. Sliding action of the apex P1 of the ratchet teeth 1411′ in the opposite direction L against the lateral side of the stationary teeth 143′ over the apex P2 of the stationary teeth 143′ results in overcoming the friction force between the attracting section 152′ of the magnetic member 150′ and the driving roller 141′, where the apex P1 of the ratchet teeth 1411′ is oriented toward the top side 1433′ of an adjacent stationary teeth 143′. Note that at this time, the biasing unit 139′ is still on the way to its initial shape or has not fully retrieved to its initial position.

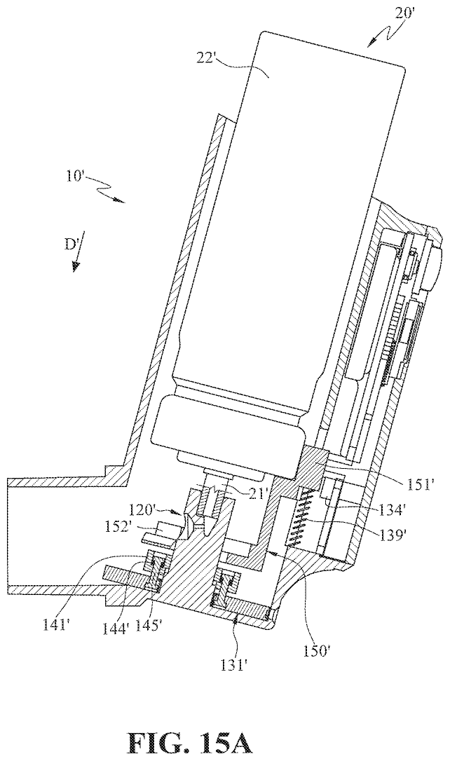

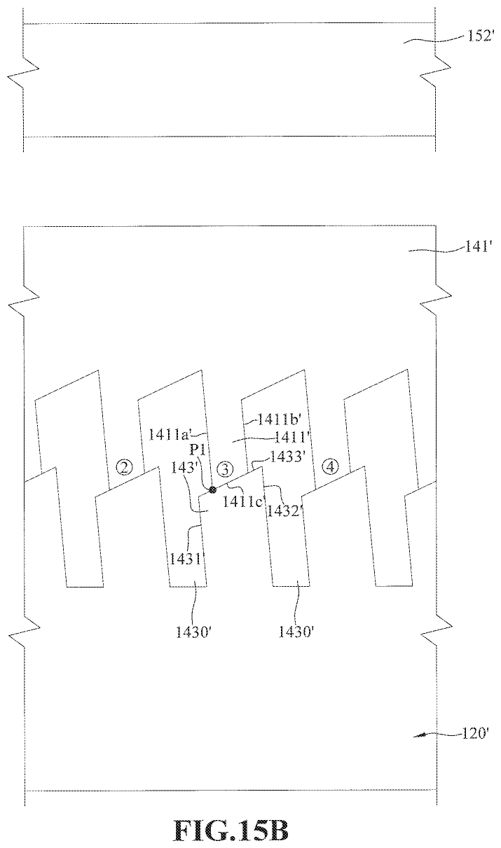

Referring to FIGS. 15A and 15B, during the further recovery of its initial position of the biasing unit 139′ in the opposite direction L results in simultaneous moving of the magnetic member 150′, thereby disposing the sensing section 151 to the first sensing position of the reed switch 134′, where the attracting section 152′ disengages the driving roller 141′ owing to the limited spacing of the driving roller 141′ from the marked wheel 131′ due to presence of the spring units 144′ between the driving roller 141′ and the marked wheel 131′, where the apex P1 of the ratchet teeth 1411′ is oriented toward the top side 1433′ of an adjacent stationary teeth 143′ such that the bottom side 1411c′ of the ratchet teeth 1411′ abuts against the top side the top side 1433′ of the adjacent stationary teeth 143′ prior to dropping into an adjacent gap 1430′ defined between an adjacent pair of the stationary teeth 143′, as best shown in FIG. 15b.

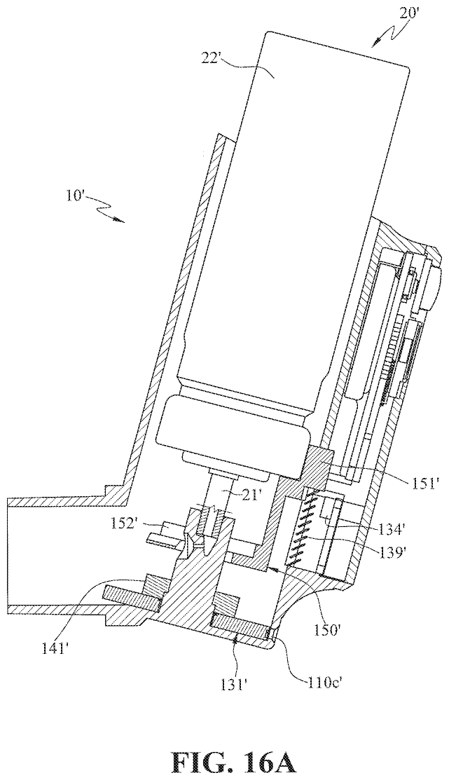

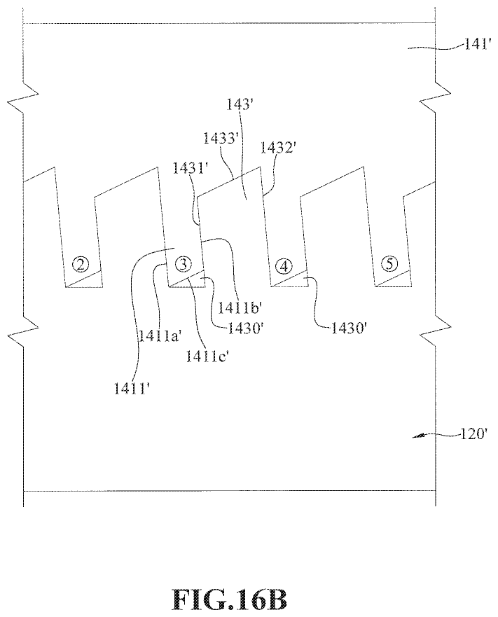

FIGS. 16A and 16B respectively show a state, in which the spray mouth 21′ is shifted relative to the bottle body 22′ from the retracted position to the normal position due to full recovery of the biasing unit 139′ to its initial position, where the sensing section 151′ of the magnetic member 150′ is disposed from the second position to the first sensing position of the reed switch 134′ and where the attracting section 152′ is spaced limitedly apart from the driving roller 141′. As best shown in FIG. 16B, since the bottom side 1411c′ of the ratchet teeth 1411′ is oriented and slides toward an adjacent one of the gap 1430′ of adjacent pair of the stationary teeth 143′, thereby driving the marked wheel 131′ to rotate in a preset angle and displaying a specific one of the marked pattern 1313′ via the viewing window 110c′ (see FIG. 11). Note that the specific one of the marked patterns 1313′ defining the discharged number of the substance sprayed out from the spray mouth 21′ of the bottle body 22′ possess one more spray action compared with the preceding discharged number.

FIGS. 16A and 16B respectively show the second embodiment of the present invention, since the electronic or mechanical elements are employed to count the discharged number of the substance from the spray bottle 20′, the marked patterns 1313′ of the spray bottle 20′ can be visible via the viewing window 110c′ in the casing 110′, thereby understanding the discharged number of the spray bottle 20′. In the event that the information of the electronic inhaling device 10′ of the present invention is required to be transmitted to an electronic device, like a smart phone of a user, the user can press the button 137′ to activate the communication device 136′, which transmits the information of the spray bottle 20′ to the smart phone. Preferably, the smart phone has a monitoring program utilized by the communication unit 136′ for transmitting the information of the spray bottle 20′. The information preferably includes the discharged number of the substance sprayed out from the spray mouth 21′ of the bottle body 22′, the remaining content in the bottle 20′ and a schedule time for replacing the spray bottle 20′ with a new one.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1-26. (canceled)

27. A counting device for counting the number of actuations of a cylindrical canister, the cylindrical canister having a first end upon which a user presses to actuate, and a second end within which a dispensing stem is disposed along a vertical axis, the counting device comprising:
a gear comprising an end surface, with numerical notations disposed on the end surface;
an actuation arm disposed along the vertical axis and positioned to interact with the second end of the canister as the canister actuates, such that movement of the canister along the vertical axis is translated to movement of the actuation arm along the same vertical axis, wherein the actuation arm is coupled to the gear to cause the gear to rotate when the actuation arm is translated along the vertical axis; and
a viewing window disposed over the end surface of the gear, limiting visibility of the numerical notations to a notation representative of the number of actuations of the cylindrical canister.
